(19) United States
(12) Patent Application Publication
(10) Pub. No.: US 2010/0114305 A1
(43) Pub. Date: May 6, 2010

(54) Implantable Valvular Prosthesis

(76) Inventors: Wei-Chang Kang, Tainan City (TW); Yu-Li Kang, Tainan City (TW)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 12/261,255

(22) Filed: Oct. 30, 2008

(51) Int. Cl. A61F 2/24; A61F 2/06

(52) U.S. Cl. 623/2.1; 623/1.26

(57) ABSTRACT

An implantable valvular prosthesis is adapted for implantation in a position of an anatomic valve that controls passage of blood flowing from a ventricle to an artery in a patient's heart. The implantable valvular prosthesis includes a tubular stent body and at least one valve flap member.

The tubular stent body includes a valve exclusion region and a tubular flare region.

### BACKGROUND OF THE INVENTION

1. Field of the Invention

This invention relates to a prosthesis, more particularly to an implantable valvular prosthesis.

2. Description of the Related Art

Various artificial heart valves for replacing anatomic valves are available in the market and are capable of curing valvular heart diseases that arise from valvular insufficiency or stenosis. Referring to FIG. 1, US2006/0167543 discloses a valvular prosthesis 20 for replacing an anatomic valve such as an aortic valve 101 that has three cusp portions. The valvular prosthesis 20 surrounds a longitudinal axis and includes a stent body member, a graft, and valve flaps. The stent body member includes a distal anchor section 201, an intermediate annular section 203, a transitional section 202 that interconnects the distal anchor section 201 and the intermediate annular section 203, and that extends downward from the distal anchor section 201 to the intermediate annular section 203 and inwardly toward the longitudinal axis, and a proximal anchor flange that projects radially and away from the longitudinal axis. The valvular prosthesis 20 is percutaneously delivered to a position of the aortic valve 101 that controls passage of blood flowing from a left ventricle 103 to an aorta 100 by virtue of cardiac catheterization. Subsequently, the valvular prosthesis 20 is deployed at the position of the aortic valve 101 in order to press each of the cusp portions of the aortic valve 101 against the aorta 100 and thereby replace the aortic valve 101.

The artery (the aorta 100 in this prior art) has a luminal wall that has a tubular juncture region to which a base portion of the anatomic valve (the aortic valve 101 in this prior art) is connected, a tubular proximal region adjacent to the tubular juncture region, and a tubular distal region adjacent to the tubular proximal region and opposite to the tubular juncture region. The tubular juncture region, the tubular proximal region, and the tubular distal region cooperatively define an inner convex surface of a sinus of Valsalva 102. The ventricle (the left ventricle 103 in this prior art) has a tubular bordering region that confronts a chamber of the ventricle, that is adjacent to the tubular juncture region, and that is upstream of the tubular proximal region and the tubular distal region in terms of an ejected flow of blood under systole pressure.

When the valvular prosthesis 20 is deployed at the position of the aortic valve 101, the distal anchor section 201, the transitional section 202, and the intermediate annular section 203 cooperatively press only some parts of each of the cusp portions against the tubular proximal region. Since the shape of the transitional section 202 is not conformable to the shape of the tubular proximal region, the transitional section 202 is incapable of abutting against the tubular proximal region and pressing each of the cusp portions entirely against the inner convex surface of the sinus of Valsalva 102. Consequently, a space 104 between each of the cusp portions and the inner convex surface of the sinus of Valsava 102 is formed. Blood may flow into the space 104 and be trapped therein, thereby resulting in thrombus.

Since the shape of the distal anchor section 201 is not conformable to the shape of the tubular distal region, the distal anchor section 201 is unable to abut against the tubular distal region therealong. Hence immobility of the distal anchor section 201 in the aorta 100 cannot be insured. Particularly, when the blood flows from the left ventricle 103 to the aorta 100, the valvular prosthesis 20 may further be shaken to shift from the position of the aortic valve 101 as a result of impact by a surge of the blood under the systole pressure.

### SUMMARY OF THE INVENTION

The object of the present invention is to provide an implantable valvular prosthesis that can overcome the aforesaid drawbacks of the prior art.

According to this invention, an implantable valvular prosthesis is adapted for implantation in a position of an anatomic valve that controls passage of blood flowing from a ventricle to an artery in a patient's heart. The artery has a luminal wall that has a tubular juncture region to which a base portion of the anatomic valve is connected, a tubular proximal region adjacent to the tubular juncture region, and a tubular distal region adjacent to the tubular proximal region and opposite to the tubular juncture region. The ventricle has a tubular bordering region that confronts a chamber of the ventricle, that is adjacent to the tubular juncture region, and that is upstream of the tubular proximal region and the tubular distal region in terms of an ejected flow of the blood under systole pressure. The implantable valvular prosthesis includes a tubular stent body and at least one valve flap member.

The tubular stent body is made from a material expandable at a site of implantation, and has luminal and abluminal surfaces, and a central tubular opening along a central longitudinal axis of the tubular stent body. The tubular stent body includes a valve exclusion region and a tubular flare region.

The valve exclusion region is configured to be of a dimension when the tubular stent body is expanded, such that during systole, the valve exclusion region is kept in intimate contact with the tubular juncture region while pressing the base portion of the anatomic valve to deflect a cusp portion of the anatomic valve to orient along the central longitudinal axis.

The tubular flare region is divergent from the valve exclusion region towards the artery, terminates at a cusp-concealing waistline that is brought in intimate contact with the luminal wall of the artery so as to ensure concealing of the cusp portion from exposure to a lumen of the artery, and is configured such that the tubular flare region is brought to abut against the tubular proximal region along the central longitudinal axis so as to ensure the concealing of the cusp portion of the anatomic valve when the tubular juncture region is distended as a result of the systole pressure, and such that the tubular flare region counteracts a biasing force of the cusp portion of the anatomic valve. A profile of the tubular flare region is able to ensure immobility of the tubular stent body relative to the tubular juncture region.

The valve flap member is disposed to associate with the tubular flare region, extends from the luminal surface of the tubular flare region of the tubular stent body towards the central longitudinal axis, and is biased to a closed position under zero pressure differential across the implantable valvular prosthesis. The closed position acts to interrupt a passage of the blood that flows through the central tubular opening.

### BRIEF DESCRIPTION OF THE DRAWINGS

Other features and advantages of the present invention will become apparent in the following detailed description of the preferred embodiments of this invention, with reference to the accompanying drawings, in which:

FIG. 1 is a schematic view illustrating the situation where a valvular prosthesis disclosed in US2006/0167543 is implanted in a sinus of Valsalva;

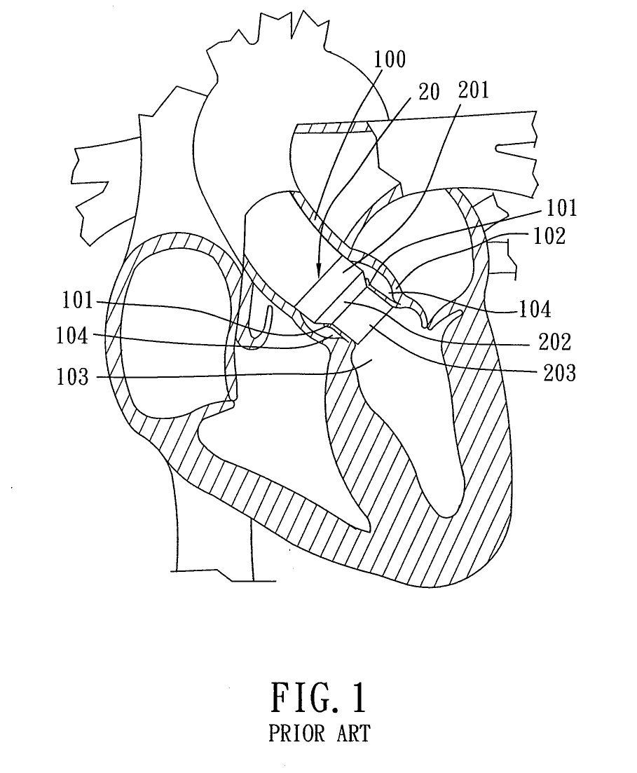

FIG. 2 is a schematic view of the first preferred embodiment of an implantable valvular prosthesis according to this invention;

FIG. 3 is a schematic view illustrating a tubular stent body according to the first preferred embodiment;

FIG. 4 is a schematic view illustrating how the implantable valvular prosthesis shown in FIG. 2 is delivered to the sinus of Valsalva by virtue of a catheter according to the first preferred embodiment;

FIG. 5 is a schematic view illustrating how the implantable valvular prosthesis shown in FIG. 2 is implanted in the sinus of Valsalva according to the first preferred embodiment; and

FIG. 6 is a schematic view illustrating a tubular stent body of the second preferred embodiment of an implantable valvular prosthesis according to this invention.

### DETAILED DESCRIPTION OF THE PREFERRED EMBODIMENTS

Before the present invention is described in greater detail, it should be noted that same reference numerals have been used to denote like elements throughout the specification.

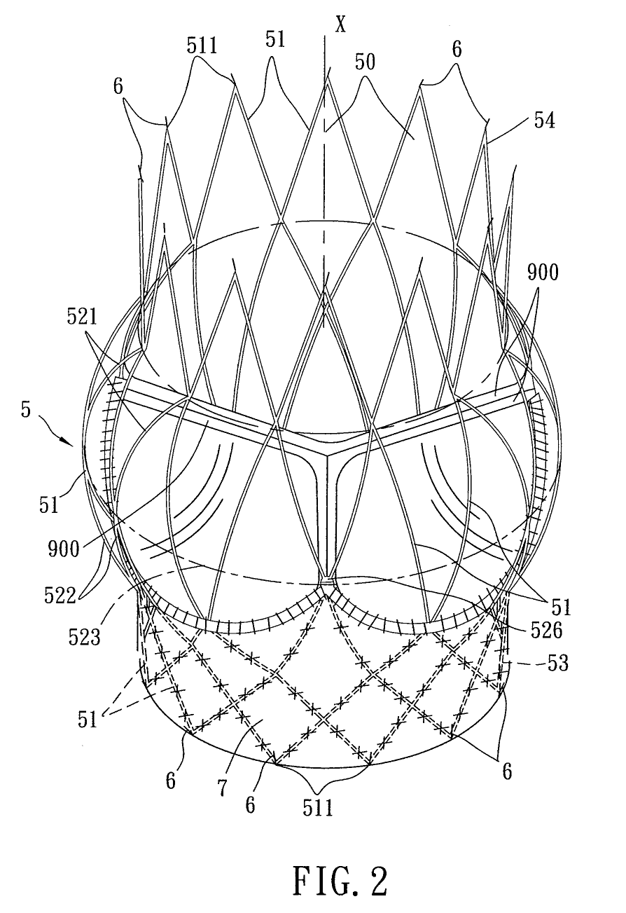

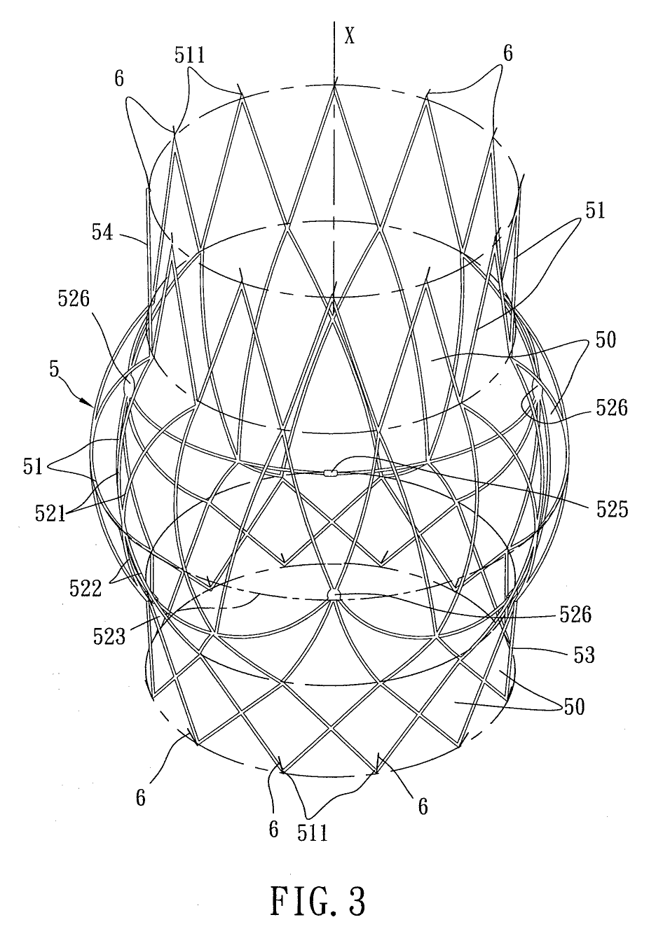

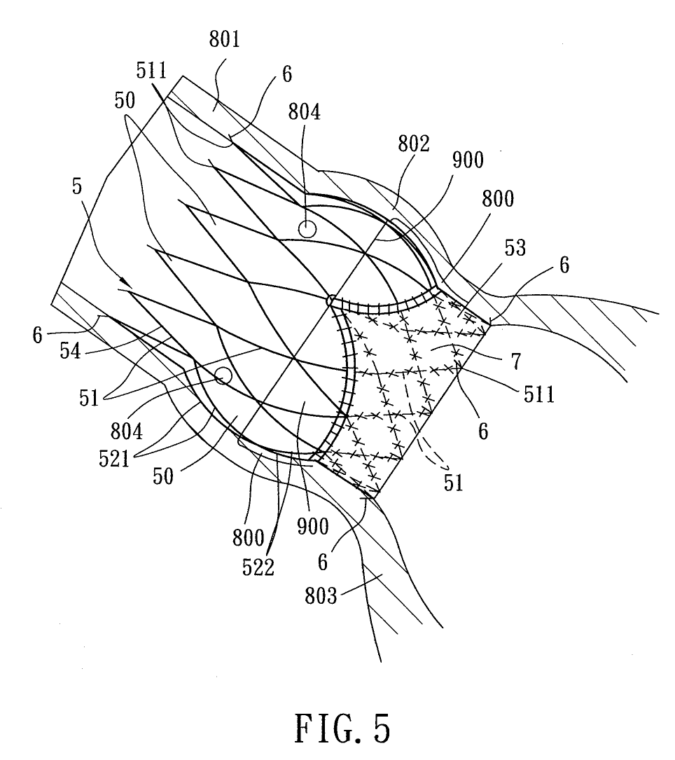

Referring to FIGS. 2, 3, and 5, the first preferred embodiment of an implantable valvular prosthesis according to the present invention is adapted for implantation in a position of an anatomic valve which controls passage of blood flowing from a ventricle to an artery in a patient's heart. In this embodiment, the implantable valvular prosthesis for the sake of illustration is shown to be implanted in a position of an aortic valve 800 that controls passage of blood flowing from a left ventricle 803 to an aorta 801. The implantable valvular prosthesis can also be implanted in a position of a pulmonary valve that controls passage of blood flowing from a right ventricle to pulmonary arteries (not shown). The implantable valvular prosthesis includes a tubular stent body 5 and three valve flap members 900.

The tubular stent body 5 is made from a material expandable at a site of implantation, and has luminal and abluminal surfaces, and a central tubular opening along a central longitudinal axis (X) of the tubular stent body 5. In this embodiment, the tubular stent body 5 is made from a plurality of metal struts 51 that are made from a shape memory material, that intersect with each other, and that cooperatively define a plurality of through-holes 50 each in a shape of a rhombus. It is noted that the shape of the through-holes 50 is not limited to the rhombus in other embodiments of the invention.

The tubular stent body 5 includes a valve exclusion region 53 and a tubular flare region 522. The valve exclusion region 53 has a plurality of apexes 511 that are formed by the intersections of the metal struts 51 and a plurality of the through-holes 50. The valve exclusion region 53 is configured to be of a dimension when the tubular stent body 5 is expanded, such that during systole, the valve exclusion region 53 is kept in intimate contact with a tubular juncture region of a luminal wall of the aorta 801 while pressing a base portion of the aortic valve 800 to deflect a cusp portion of the aortic valve 800 to orient along the central longitudinal axis (X).

The tubular flare region 522 is divergent from the valve exclusion region 53 towards the aorta 801, and terminates at a cusp-concealing waistline 523 that is brought in intimate contact with the luminal wall of the aorta 801 so as to ensure concealing of the cusp portion from exposure to a lumen of the aorta 801. The tubular flare region 522 is capable of abutting against a tubular proximal region of the luminal wall of the aorta 801 and of pressing the aortic valve 800 entirely against the inner convex surface of the sinus of Valsalva 802 because the shape of the tubular flare region 522 is conformable to the shape of the tubular proximal region. Furthermore, the tubular flare region 522 is configured such that the tubular flare region 522 is brought to abut against the tubular proximal region along the central longitudinal axis (X) so as to ensure the concealing of the cusp portion of the aortic valve 800 when the tubular juncture region is distended as a result of the systole pressure, and such that the tubular flare region 522 counteracts a biasing force of the cusp portion of the aortic valve 800. Thus, during the systole or the diastole, there is little or no space formed between each of the cusp portions and the inner convex surface of the sinus of Valsalva 802, thereby alleviating the possibility of thrombosis.

The tubular stent body 5 further includes a tubular tapering region 521 that is convergent from the cusp-concealing waistline 523 towards the aorta 801. The tubular tapering region 521 is capable of abutting against a tubular distal region of the luminal wall of the aorta 801 because the shape of the tubular tapering region 521 is conformable to the shape of the tubular distal region. Furthermore, the tubular tapering region 521 is configured such that the tubular tapering region 521 is brought to abut against the tubular distal region along the central longitudinal axis (X) when the tubular juncture region is distended as a result of the systole pressure. When the blood flowing from the left ventricle 803 to the aorta 801 impacts against the tubular tapering region 521, the implantable valvular prosthesis could stand unmoved at the position of the aortic valve 800.

The tubular stent body 5 further includes an artery anchor region 54 that extends from the tubular tapering region 521, that has a plurality of apexes 511 formed by the intersections of the metal struts 51 and a plurality of the through-holes 50, and that is configured to be of a dimension when the tubular stent body 5 is expanded, such that during the systole, the artery anchor region 54 is kept in intimate contact with the luminal wall of the aorta 801.

In this embodiment, the valve exclusion region 53, the tubular flare region 522, the tubular tapering region 521, and the artery anchor region 54 are formed integrally.

Preferably, the ratio of the external diameter pertaining to the cusp-concealing waistline 523 to the external diameter pertaining to the artery anchor region 54 is 1.3 to 1. The ratio of the external diameter pertaining to the cusp-concealing waistline 523 to the external diameter pertaining to the valve exclusion region 53 is 1.3 to 1 as well. These two ratios correspond to the ratio of the maximum internal diameter pertaining to the sinus of Valsalva 802 to the internal diameter pertaining to the aorta 801. Consequently, the tubular stent body 5 can be securely installed in the patient's heart. It is noted that the external diameters of the cusp-concealing waistline 523, the valve exclusion region 53, and the artery anchor region 54 can be modified in order to correspond to different ratios of the maximum internal diameter pertaining to the sinus of Valsalva 802 to the internal diameter pertaining to the aorta 801.

The valve flap members 900 which are able to replace the aortic valve 800, and which are disposed to associate with the tubular flare region 522, extend from the luminal surface of the tubular flare region 522 of the tubular stent body 5 towards the central longitudinal axis (X) The valve flap members 900 are biased to a closed position under zero pressure differential across the implantable valvular prosthesis. The closed position acts to interrupt a passage of the blood that flows through the central tubular opening. The valve flap members 900 are able to replace the pulmonary valve (not shown) when the implantable valvular prosthesis is implanted in the position of the pulmonary valve.

In this embodiment, the valve flap members 900 are sewn to the tubular flare region 522. The tubular flare region 522 has a plurality of first positioning members 525 that are spaced apart, and a plurality of second positioning members 526 that are spaced apart and that are circular. The first and second positioning members 525, 526 are suitable for orienting the implantable valvular prosthesis to a correct position relative to the aortic valve 800. Preferably, the first and second positioning members 525, 526 are made from a radiopaque material. It is noted that the first and second positioning members 525, 526 can be omitted in other embodiments.

The implantable valvular prosthesis further includes a graft member 7 which covers the abluminal surface of the valve exclusion region 53 of the tubular stent body 5 so as to ensure that the central tubular opening is a sole available route for the ejected flow of the blood under the systole pressure during the systole. In this embodiment, the graft member 7 covers a portion of the abluminal surface of the tubular flare region 522 of the tubular stent body 5 as well. It is noted that the graft member 7 can be applied to other regions of the tubular stent body 5 when needed.

The implantable valvular prosthesis further includes a plurality of anchoring needles 6 which are configured when the tubular stent body 5 is expanded, such that the anchoring needles 6 are brought to engage the luminal wall of the aorta 801 and to anchor thereat. In this embodiment, the anchoring needles 6 extend upwardly and obliquely from the apexes 511 of the valve exclusion region 53 and the artery anchor region 54.

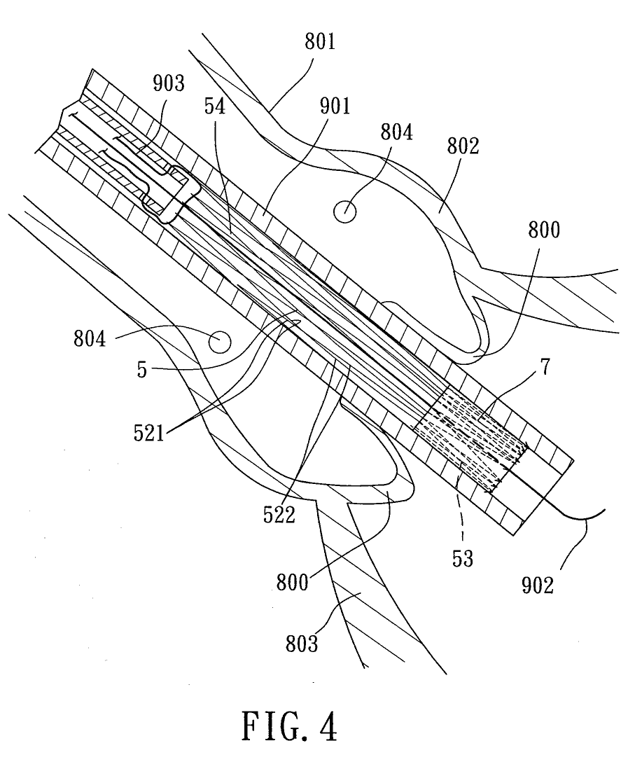

Referring to FIGS. 2, 4, and 5, a pulling string 903 extends through the through-holes 50 of the artery anchor region 54. A guide wire 902 extends longitudinally through the tubular stent body 5. By virtue of the pulling string 903 and the guide wire 902, the tubular stent body 5 can be stretched in a direction along the central longitudinal axis (X) and contracted inwardly toward the central longitudinal axis (X). Thus, the tubular stent body 5 can be placed into a catheter 901. Since the feature of the invention does not reside in a method of placing the tubular stent body 5 into the catheter 901, further details of the same are omitted herein for the sake of brevity.

During cardiac catheterization, after one end of the catheter 901 is delivered to the sinus of Valsalva 802 and passed through the aortic valve 800, the valve exclusion region 53, the tubular flare region 522, the tubular tapering region 521, and the artery anchor region 54 are withdrawn from the catheter 901 in sequence. The valve exclusion region 53 withdrawn from the catheter 901 is expanded to intimately contact the tubular juncture region. The anchoring needles 6 on the apexes 511 of the valve exclusion region 53 are brought to pierce upwardly and obliquely into the luminal wall of the aorta 801. Afterward, the tubular flare region 522 withdrawn from the catheter 901 is expanded to abut against the tubular proximal region of the luminal wall of the aorta 801. Subsequently, the tubular tapering region 521 withdrawn from the catheter 901 is expanded to abut against the tubular distal region of the luminal wall of the aorta 801. Two of the through-holes 50 of the tubular tapering region 521 are oriented to a position such that each of the two through-holes 50 is in spatial communication with a respective one of coronary arteries 804. Finally, the artery anchor region 54 withdrawn from the catheter 901 is expanded to intimately contact the luminal wall of the aorta 801.

Since the tubular stent body 5 abuts against the luminal wall of the aorta 801 along its length, the implantable valvular prosthesis is securely immobilized at the position of the aortic valve 800 even though the blood flowing from the left ventricle 803 to the aorta 801 impacts against the implantable valvular prosthesis.

When the tubular stent body 5 is not positioned at a desired place, the tubular stent body 5 can be pulled back into the catheter 901 via the pulling string 903 that can drag the apexes 511 of the artery anchor region 54 before the artery anchor region 54 is entirely withdrawn from the catheter 901. Therefore, the position of the tubular stent body 5 can be adjusted when needed.

A chest X-ray is suitable for determination of the position of the implantable valvular prosthesis. After the cardiac catheterization, the relative position between the first positioning members 525, the second positioning members 526, and the heart can be revealed by way of a chest radiograph.

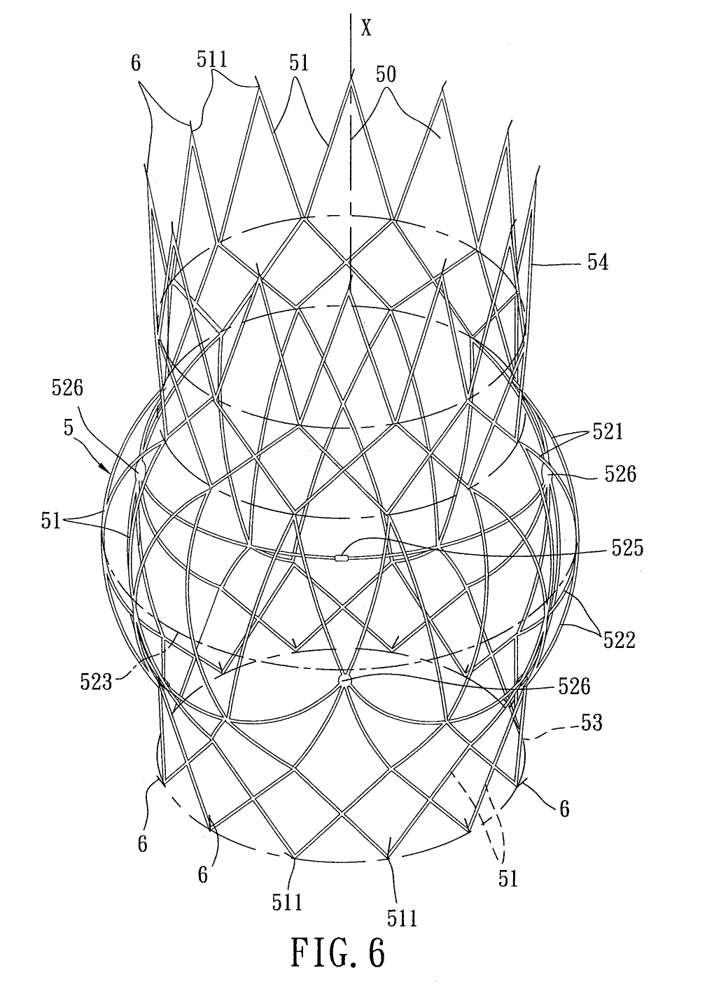

Referring to FIG. 6, the second preferred embodiment of the implantable valvular prosthesis according to this invention is illustrated. The structure of this preferred embodiment is similar to the structure of the first preferred embodiment. The difference between this preferred embodiment and the first preferred embodiment resides in that the artery anchor region 54 in the second preferred embodiment is longer than that of the first preferred embodiment, thereby resulting in enhanced secure installation of the implantable valvular prosthesis at the implantation site.

While the present invention has been described in connection with what are considered the most practical and preferred embodiments, it is understood that this invention is not limited to the disclosed embodiments but is intended to cover various arrangements included within the spirit and scope of the broadest interpretation and equivalent arrangements.

What is claimed is:

1. An implantable valvular prosthesis adapted for implantation in a position of an anatomic valve which controls passage of blood flowing from a ventricle to an artery in a patient's heart, the artery having a luminal wall that has a tubular juncture region to which a base portion of the anatomic valve is connected, a tubular proximal region adjacent to the tubular juncture region, and a tubular distal region adjacent to the tubular proximal region and opposite to the tubular juncture region, the ventricle having a tubular bordering region confronting a chamber of the ventricle, adjacent to the tubular juncture region, and upstream of the tubular proximal region and the tubular distal region in terms of an ejected flow of the blood under systole pressure, said implantable valvular prosthesis comprising:
a tubular stent body which is made from a material expandable at a site of implantation, and which has luminal and abluminal surfaces, and a central tubular opening along a central longitudinal axis of said tubular stent body, said tubular stent body including
a valve exclusion region which is configured to be of a dimension when said tubular stent body is expanded, such that during systole, said valve exclusion region is kept in intimate contact with the tubular juncture region while pressing the base portion of the anatomic valve to deflect a cusp portion of the anatomic valve to orient along the central longitudinal axis, and
a tubular flare region which is divergent from said valve exclusion region towards the artery, which terminates at a cusp-concealing waistline brought in intimate contact with the luminal wall of the artery so as to ensure concealing of the cusp portion from exposure to a lumen of the artery, and which is configured such that said tubular flare region is brought to abut against the tubular proximal region along the central longitudinal axis so as to ensure the concealing of the cusp portion of the anatomic valve when the tubular juncture region is distended as a result of the systole pressure, and such that said tubular flare region counteracts a biasing force of the cusp portion of the anatomic valve, a profile of said tubular flare region being able to ensure immobility of said tubular stent body relative to the tubular juncture region; and
at least one valve flap member disposed to associate with said tubular flare region, extending from said luminal surface of said tubular flare region of said tubular stent body towards the central longitudinal axis, and biased to a closed position under zero pressure differential across said implantable valvular prosthesis, the closed position acting to interrupt a passage of the blood that flows through said central tubular opening.

2. The implantable valvular prosthesis as claimed in claim 1, wherein said tubular stent body further includes a tubular tapering region which is convergent from said cusp-concealing waistline towards the artery and which is configured such that said tubular tapering region is brought to abut against the tubular distal region along the central longitudinal axis when the tubular juncture region is distended as a result of the systole pressure.

3. The implantable valvular prosthesis as claimed in claim 2, wherein said tubular stent body further includes an artery anchor region that extends from said tubular tapering region and that is configured to be of a dimension when said tubular stent body is expanded, such that during the systole, said artery anchor region is kept in intimate contact with the luminal wall of the artery.

4. The implantable valvular prosthesis as claimed in claim 1, further comprising a graft member covering said abluminal surface of said valve exclusion region of said tubular stent body so as to ensure that said central tubular opening is a sole available route for the ejected flow of the blood under the systole pressure during the systole.

5. The implantable valvular prosthesis as claimed in claim 1, further comprising a plurality of anchoring needles which are configured when said tubular stent body is expanded, such that said anchoring needles are brought to engage the luminal wall of the artery and to anchor thereat.
